(19) United States
(12) Patent Application Publication
(10) Pub. No.: US 2021/0215683 A1
(43) Pub. Date: Jul. 15, 2021

(54) FIELD EFFECT TRANSISTOR-BASED BIOSENSOR FOR DETECTING WHOLE-CELL BACTERIA AND FIELD EFFECT TRANSISTOR-BASED BIOSENSOR ASSEMBLY INCLUDING THE SAME

(71) Applicant: National Chiao Tung University, Hsinchu City (TW)

(72) Inventors: Yuh-Shyong Yang, Hsinchu City (TW); Daisy Cheng, Hsinchu City (TW); Chen-Yun Lin, Hsinchu City (TW)

(73) Assignee: National Chiao Tung University

(21) Appl. No.: 16/741,831

(22) Filed: Jan. 14, 2020

Related U.S. Application Data
(60) Provisional application No. 62/793,974, filed on Jan. 18, 2019.

(51) Int. Cl. G01N 33/543; G01N 27/414

(52) U.S. Cl. CPC G01N 33/5438; G01N 27/4145

(57) ABSTRACT

Disclosed is a field effect transistor-based biosensor for detecting whole-cell bacteria which includes a source, a drain, and a biosensing member disposed between the source and the drain. The biosensing member includes at least one semiconductor wire, a surface modification layer, and a plurality of detecting elements. The semiconductor wire serves as a semiconductor channel interconnecting the source and the drain, and has a length so as to permit the biosensing member to capture the whole-cell bacteria. Also disclosed is a field effect transistor-based biosensor assembly including the biosensor.

### CROSS-REFERENCE TO RELATED APPLICATION

This application claims priority of U.S. Provisional Application No. 62/793,974, filed on Jan. 18, 2019, which is incorporated by reference herein in its entirety.

### FIELD

The disclosure relates to a field effect transistor-based biosensor, and more particularly to a field effect transistor-based biosensor for detecting whole-cell bacteria. The disclosure also relates to a field effect transistor-based biosensor assembly including the field effect transistor-based biosensor.

### BACKGROUND

Detection of bacterial pathogens is of utmost importance in various fields, which include food and medical industry, public health, social security, and etc. Contamination of pathogenic bacteria in food products, medical supplies, or water sources might lead to severe consequences. For example, if a human population gets into contact with a contaminated source such as bacterial pathogens, it may cause an outbreak of bacterial infection, which is one of the common causes of morbidity and mortality. Therefore, rapid detection of bacterial pathogens is crucial for restricting the outbreak of bacterial infection. The faster the detection rate, the more the response time available to take control of the outbreak, and the sooner infected patients are treated.

Conventional bacterial pathogen detection methods include a culture screening method, a polymerase chain reaction method, an immunology-based method, and etc. Although these conventional detection methods allow the detection of single bacteria, amplification of the detected signal is required. The conventional detection methods also require culturing a single cell into a colony of cells, which is time consuming, often taking up to 72 hours. Moreover, the conventional detection methods are limited to be executed in a specialized laboratory and require trained personnel. In addition, in order to shorten detection time and simplify testing procedures, direct detection of whole cells of the bacterial pathogens is favored over detection of biomolecules thereof, as the latter requires additional purification steps which prolong testing time, and thus adding to cost.

### SUMMARY

Therefore, an object of the disclosure is to provide a biosensor which is capable of detecting whole-cell bacteria.

According to a first aspect of the disclosure, there is provided a field effect transistor-based biosensor for detecting whole-cell bacteria. The field effect transistor-based biosensor includes a source, a drain spaced apart from the source in a first direction, and a biosensing member disposed between the source and the drain. The biosensing member includes at least one semiconductor wire, a surface modification layer, and a plurality of detecting elements. The at least one semiconductor wire serves as a semiconductor channel interconnecting the source and the drain, and has a length in the direction so as to permit the biosensing member to capture the whole-cell bacteria. The surface modification layer is formed on the semiconductor wire. The detecting elements bond to the surface modification layer and are capable of capturing the whole-cell bacteria.

According to a second aspect of the disclosure, there is provided a field effect transistor-based biosensor assembly for detecting whole-cell bacteria. The field effect transistor-based biosensor assembly includes a plurality of the biosensors of the first aspect of the disclosure which are displaced from one another.

### BRIEF DESCRIPTION OF THE DRAWINGS

Other features and advantages of the disclosure will become apparent in the following detailed description of the embodiments with reference to the accompanying drawings, of which:

FIG. 1 is a schematic view of a first embodiment of a field effect transistor-based biosensor for detecting whole-cell bacteria according to the disclosure;

FIG. 2 is a schematic planar view of the first embodiment of the field effect transistor-based biosensor for detecting whole-cell bacteria according to the disclosure;

FIG. 3 is a diagram illustrating a reaction scheme for forming a surface modification layer included in the first embodiment of the field effect transistor-based biosensor for detecting whole-cell bacteria according to the disclosure;

FIG. 4 is a schematic planar view of a second embodiment of a field effect transistor-based biosensor for detecting whole-cell bacteria according to the disclosure;

FIG. 5 is an exploded schematic perspective view of a first embodiment of a field effect transistor-based biosensor assembly for detecting whole-cell bacteria according to the disclosure;

FIG. 6 is a schematic planar view of the first embodiment of the field effect transistor-based biosensor assembly for detecting whole-cell bacteria according to the disclosure;

FIG. 7 is a schematic graph illustrating the determination of the whole-cell bacteria concentration based on the detection result obtained from the field effect transistor-based biosensor assembly according to the disclosure;

FIG. 8 is an exploded schematic perspective view of a second embodiment of a field effect transistor-based biosensor assembly for detecting whole-cell bacteria according to the disclosure;

FIG. 9 is a schematic planar view of a third embodiment of a field effect transistor-based biosensor assembly for detecting whole-cell bacteria according to the disclosure; and

FIG. 10 is a schematic planar view of a fifth embodiment of a field effect transistor-based biosensor assembly for detecting whole-cell bacteria according to the disclosure.

### DETAILED DESCRIPTION

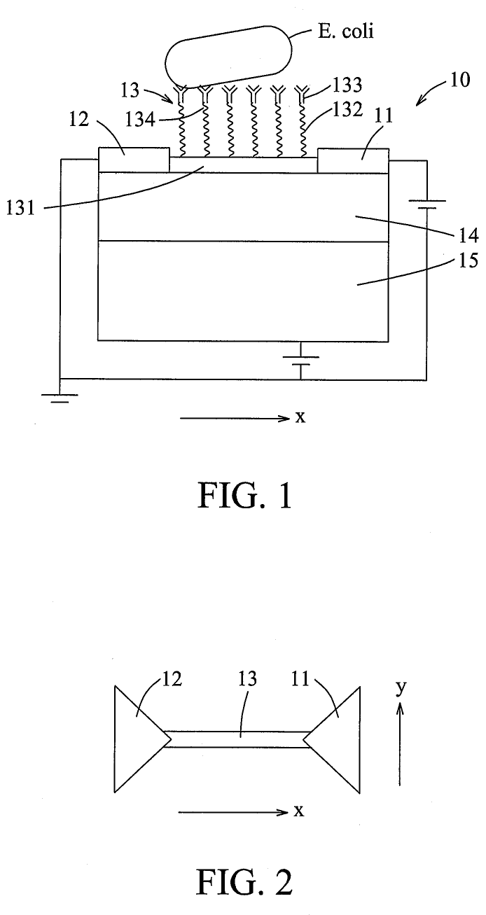

Referring to FIGS. 1 and 2, a first embodiment of a field effect transistor-based biosensor 10 for detecting whole-cell bacteria according to the disclosure includes a source 11, a drain 12 spaced apart from the source 11 in a first direction (x), and a biosensing member 13 disposed between the source 11 and the drain 12.

The biosensing member 13 includes one semiconductor wire 131, a surface modification layer 132, and a plurality of detecting elements 133.

The semiconductor wire 131 serves as a semiconductor channel interconnecting the source 11 and the drain 12, and has a length in the first direction (x) so as to permit the biosensing member 13 to capture the whole-cell bacteria. In certain embodiments, the length of the semiconductor wire 131 is in a range from 1 μm to 5 μm. The semiconductor wire 131 further has a width in a second direction (y) transverse to the first direction (x). In certain embodiments, the width ranges from 100 nm to 400 nm. In certain embodiments, the semiconductor wire 131 has a length of 1.6 μm and a width of 100 nm.

In certain embodiments, the semiconductor wire 131 is made from a material, such as polycrystalline silicon, monocrystalline silicon, hafnium dioxide, aluminum oxide, zirconium oxide, and lanthanum oxide, but is not limited thereto.

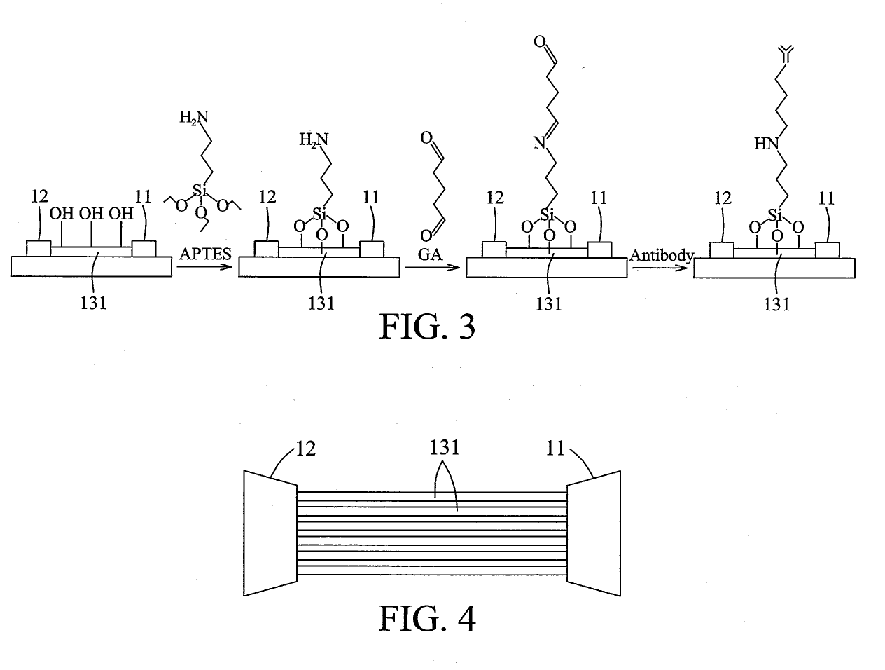

Referring to FIGS. 1 and 3, the surface modification layer 132 is formed on the semiconductor wire 131, and includes a plurality of linking moieties 134 formed distally from the semiconductor wire 131. In certain embodiments, the surface modification layer 132 is formed by the procedure described below.

Specifically, the semiconductor wire 131 is subjected to an oxygen plasma treatment, causing the surface of the semiconductor wire 131 to become more hydrophilic by forming hydroxyl groups thereon. After that, the semiconductor wire 131 is submerged in a 3-aminopropyltriethoxysilane (APTES) solution to form an amino-terminal monolayer on the surface of the semiconductor wire 131. The semiconductor wire 131 is then submerged in a glutaraldehyde (GA) solution to form the surface modification layer 132 provided with a plurality of terminal-aldehyde groups (i.e., the linking moieties 134) on the surface of the surface modification layer 132.

The detecting elements 133 are bonded to the surface modification layer 132 and are capable of capturing the whole-cell bacteria. Specifically, the detecting elements 133 are bonded to the linking moieties 134 of the surface modification layer 132, respectively. In certain embodiments, the semiconductor wire 131 formed with the surface modification layer 132 is submerged in an antibody solution so that the amines of the antibodies attach to the terminal-aldehyde groups of the GA solution, so as to immobilize the antibodies to the surface of the surface modification layer 132.

In addition to the antibodies, the detecting elements 133 may be aptamers or peptides, but are not limited thereto.

The first embodiment of the field effect transistor-based biosensor 10 further includes an isolation layer 14 for disposing the source 11, the drain 12, and the biosensing member 13 thereon, and a gate 15 disposed beneath the isolation layer 14 and electrically connected to the source 11 and the drain 12. In certain embodiments, the isolation layer 14 is made from a dielectric material.

Referring to FIG. 4, a second embodiment of a field effect transistor-based biosensor 10 for detecting whole-cell bacteria according to the disclosure is similar to the first embodiment except that, the biosensing member 13 included in the second embodiment includes a plurality of the semiconductor wires 131. In certain embodiments, the number of the the semiconductor wires 131 may be up to 40.

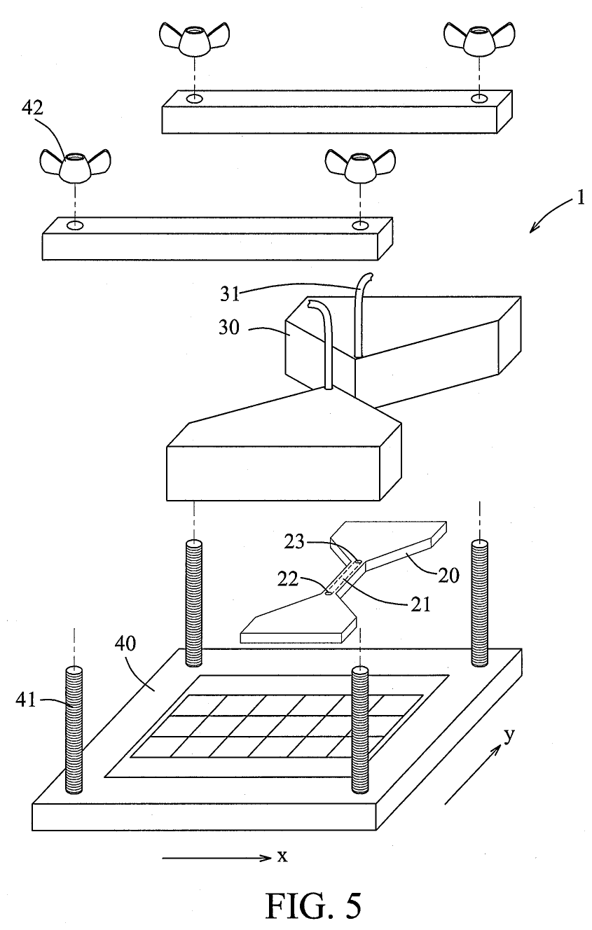

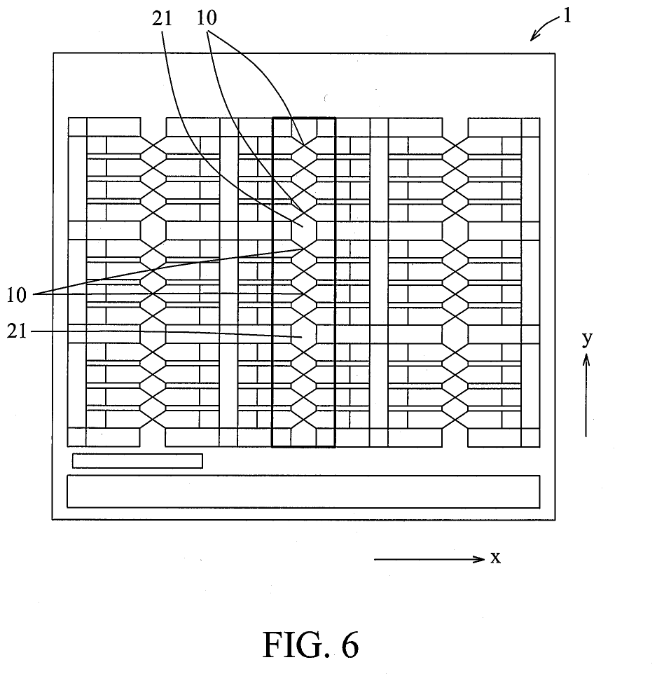

Referring to FIGS. 5 and 6, a first embodiment of a field effect transistor-based biosensor assembly 1 for detecting whole-cell bacteria according to the disclosure includes a plurality of the biosensors 10 which are displaced from one another in the second direction (y) and which are arranged in a column.

The first embodiment of the field effect transistor-based biosensor assembly 1 further includes a microfluidic member 20 and an acrylic cap 30 covering the microfluidic member 20.

The microfluidic member 20 defines a microfluidic channel 21 extending in the second direction (y) for passage of a fluid containing the bacteria therethrough, and is disposed on the biosensors 10 to permit the bacteria in the microfluidic channel 21 to access the biosensing members 13 of the biosensors 10. The microfluidic member 20 can be made from, for example, polydimethylsiloxane (PDMS) by molding. The microfluidic channel 21 has an upstream end portion and a downstream end portion. The microfluidic member 20 is formed with an inlet port 22 and an outlet port 23 disposed at the upstream end portion and the downstream end portion of the microfluidic channel 21, respectively, to fluidly communicate with the microfluidic channel 21.

The acrylic cap 30 is provided with two tubes 31 which are attached to a syringe pump (not shown). The tubes 31 are aligned with the inlet port 22 and the outlet port 23, respectively.

The first embodiment of the field effect transistor-based biosensor assembly 1 can be clamped in place on a metal platform 40 by metal bars 41 and nuts 42.

When the first embodiment of the field effect transistor-based biosensor assembly 1 is used for detecting whole-cell bacteria, a buffer is loaded using the syringe pump fora time period such that the buffer enters into one of the tubes 31, flows through the inlet port 22, the microfluidic channel 21, and the outlet port 23, and exits from the other of the tubes 31, so as to settle the field effect transistor-based biosensor assembly 1 before an ID-VG response is measured. Only after obtaining three successive overlapping drain current-gate voltage curves (ID-VG curves), the field effect transistor-based biosensor assembly 1 is deemed stable, and the last ID-VG curve is used as a baseline for the following biosensing procedure. Then, the buffer is removed from the microfluidic channel 21 by loading a biological sample to be detected using the syringe pump for a time period. The buffer is then pumped through the microfluidic channel 21 using the syringe pump for a time period to remove any unspecific binding, followed by measuring the ID-VG response for the biological sample. As mentioned above, three successive overlapping Id-Vg curves are needed before the curve serving as the signal for the biological sample can be confirmed.

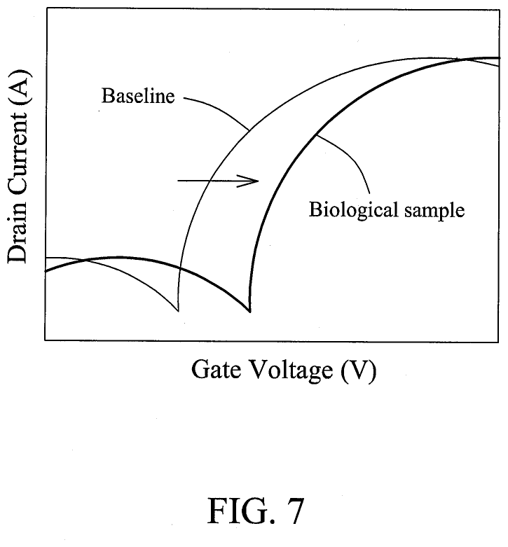

Referring to FIG. 7, the concentration of bacteria in the biological sample can be determined based on a signal difference between the Id-Vg curve serving as the base line and the Id-Vg curve obtained from measuring the biological sample, for example, based on a comparison between the threshold voltage of the Id-Vg curve serving as the base line and the threshold voltage of the Id-Vg curve obtained from measuring the biological sample.

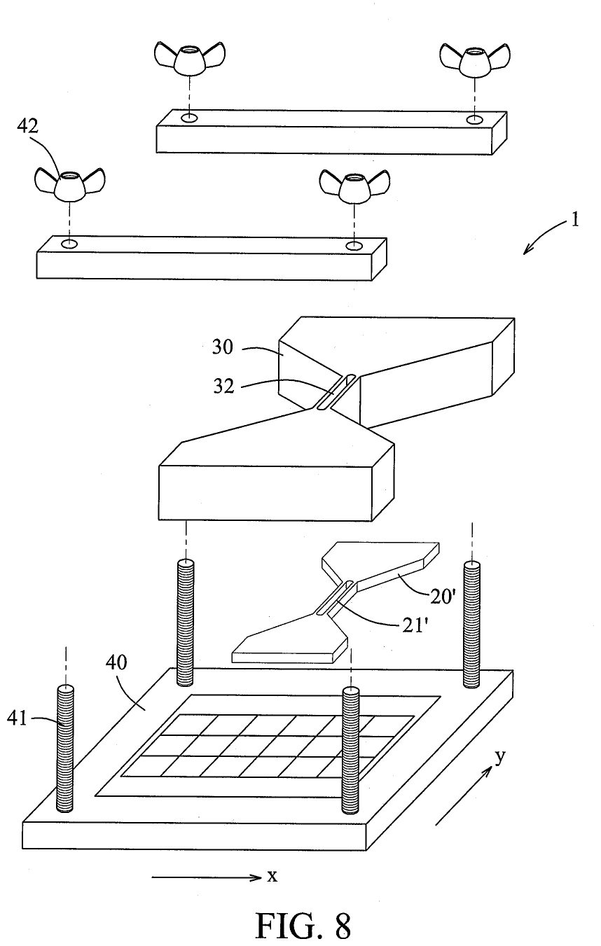

Referring to FIG. 8, a second embodiment of a field effect transistor-based biosensor assembly 1 for detecting whole-cell bacteria according to the disclosure is similar to the first embodiment, except that in the second embodiment, the microfluidic member 20 is replaced with an open-well member 20′ and that a configuration of the acrylic cap 30 in the second embodiment is different from that of the acrylic cap 30 in the first embodiment.

The open-well member 20′ defines an open well 21′ extending in the second direction (y) for accommodating a fluid that contains the bacteria therein, and is disposed on the biosensors 10 to permit the bacteria in the open well 21′ to access the biosensing members 13 of the biosensors 10.

The acrylic cap 30 in the second embodiment is provided with a groove 32 that is aligned with the open well 21′ of the open-well member 20′.

When the second embodiment of the field effect transistor-based biosensor assembly 1 is used for detecting whole-cell bacteria, the buffer or the biological sample to be detected is loaded into the open well 21′ using a pipette.

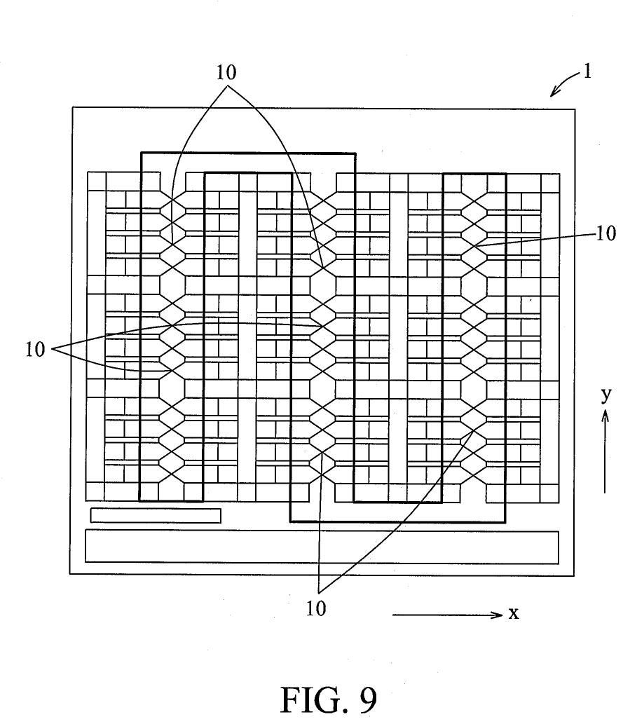

Referring to FIG. 9, a third embodiment of a field effect transistor-based biosensor assembly 1 for detecting whole-cell bacteria according to the disclosure is similar to the first embodiment, except that the biosensors 10 in the third embodiment are arranged in an array pattern, and that the microfluidic member 20 defines the microfluidic channel 21 in the form of an S-shape.

Similarly, a fourth embodiment of a field effect transistor-based biosensor assembly 1 for detecting whole-cell bacteria according to the disclosure is similar to the second embodiment, except that the biosensors 10 in the fourth embodiment are arranged in an array pattern, and that the open-well member 20′ defines the open well 21′ in the form of an S-shape.

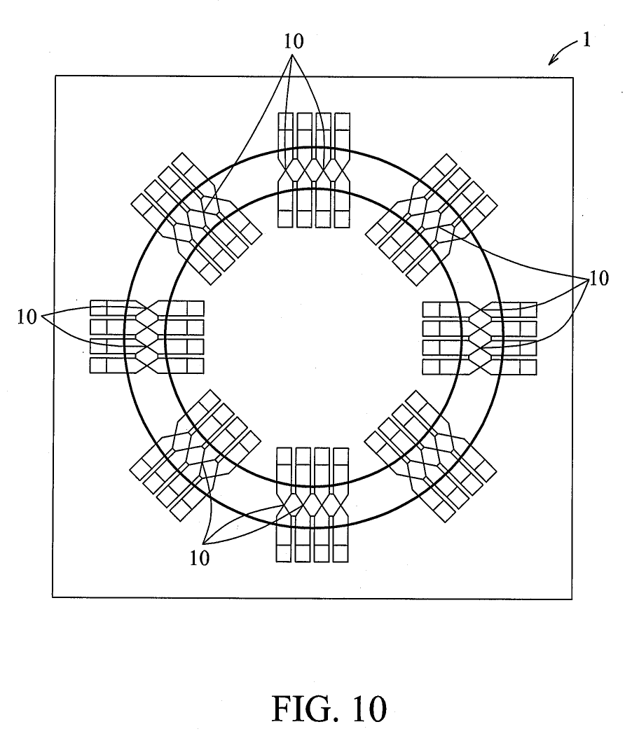

Referring to FIG. 10, a fifth embodiment of a field effect transistor-based biosensor assembly 1 for detecting whole-cell bacteria according to the disclosure is similar to the first embodiment, except that the biosensors 10 in the fifth embodiment are arranged in a circular pattern, and that the microfluidic member 20 defines the microfluidic channel 21 in the form of a circular shape.

Similarly, a sixth embodiment of a field effect transistor-based biosensor assembly 1 for detecting whole-cell bacteria according to the disclosure is similar to the second embodiment, except that the biosensors 10 in the sixth embodiment are arranged in a circular pattern, and that the open-well member 20′ defines the open well 21′ in the form of a circular shape.

In view of the aforesaid, since the semiconductor wire included in the field effect transistor-based biosensor of the disclosure has a specific length to permit the biosensing member to capture the whole-cell bacteria, and since the biosensing member includes the detecting elements which are highly sensitive and specific for the bacteria to be detected, the field effect transistor-based biosensor assembly of the disclosure can be used to detect the whole-cell bacteria in a short time period or even in real time, thus eliminating the requirement of a time-consuming cell culture procedure.

In the description above, for the purposes of explanation, numerous specific details have been set forth in order to provide a thorough understanding of the embodiments. It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects, and that one or more features or specific details from one embodiment may be practiced together with one or more features or specific details from another embodiment, where appropriate, in the practice of the disclosure.

While the disclosure has been described in connection with what are considered the exemplary embodiments, it is understood that this disclosure is not limited to the disclosed embodiments but is intended to cover various arrangements included within the spirit and scope of the broadest interpretation so as to encompass all such modifications and equivalent arrangements.

What is claimed is:

1. A field effect transistor-based biosensor for detecting whole-cell bacteria, comprising:
a source;
a drain spaced apart from said source in a first direction; and
a biosensing member disposed between said source and said drain, and including:
at least one semiconductor wire which serves as a semiconductor channel interconnecting said source and said drain and which has a length in the first direction so as to permit said biosensing member to capture the whole-cell bacteria,
a surface modification layer formed on said semiconductor wire, and
a plurality of detecting elements bonding to said surface modification layer and capable of capturing the whole-cell bacteria.

2. The field effect transistor-based biosensor according to claim 1, wherein the length of said semiconductor wire is in a range from 1 μm to 5 μm.

3. The field effect transistor-based biosensor according to claim 2, wherein said semiconductor wire further has a width ranging from 100 nm to 400 nm in a second direction transverse to the first direction.

4. The field effect transistor-based biosensor according to claim 1, wherein said semiconductor wire is made from a material selected from the group consisting of polycrystalline silicon, monocrystalline silicon, hafnium dioxide, aluminum oxide, zirconium oxide, and lanthanum oxide.

5. The field effect transistor-based biosensor according to claim 1, wherein said surface modification layer includes a plurality of linking moieties formed distally from said semiconductor wire for bonding to said detecting elements, respectively.

6. The field effect transistor-based biosensor according to claim 1, further comprising:
an isolation layer for disposing said source, said drain, and said biosensing member thereon, and
a gate disposed beneath said isolation layer and electrically connected to said source and said drain.

7. The field effect transistor-based biosensor according to claim 6, wherein said isolation layer is made from a dielectric material.

8. The biosensor device according to claim 1, wherein each of said detecting elements is selected from the group consisting of an antibody, an aptamer, and a peptide.

9. A field effect transistor-based biosensor assembly for detecting whole-cell bacteria, comprising a plurality of biosensors according to claim 1, said biosensors being displaced from one another.

10. The field effect transistor-based biosensor assembly according to claim 9, wherein said biosensors are spaced away from one another in a second direction transverse to the first direction, and are arranged in a column.

11. The field effect transistor-based biosensor assembly according to claim 9, wherein the said biosensors are arranged in an array pattern.

12. The field effect transistor-based biosensor assembly according to claim 9, wherein said biosensors are arranged in a circular pattern.

13. The field effect transistor-based biosensor assembly according to claim 10, further comprising a microfluidic member which defines a microfluidic channel extending in the second direction for passage of a fluid containing the bacteria therethrough, and which is disposed on said biosensors to permit the bacteria in the microfluidic channel to access said biosensor member.

14. The field effect transistor-based biosensor assembly according to claim 13, wherein said microfluidic channel has an upstream end portion and a downstream end portion, said microfluidic member being formed with an inlet port and an outlet port disposed at said upstream end portion and said downstream end portion of said microfluidic channel, respectively, to fluidly communicate with said microfluidic channel.

15. The field effect transistor-based biosensor assembly according to claim 10, further comprising an open-well member which defines an open well extending in the second direction for accommodating a fluid that contains the bacteria therein, and which is disposed on said biosensors to permit the bacteria in said open well to access said biosensor member.

16. The field effect transistor-based biosensor assembly according to claim 11, further comprising a microfluidic member which defines a microfluidic channel in the form of an S-shape for passage of a fluid containing the bacteria therethrough, and which is disposed on said biosensors to permit the bacteria in said microfluidic channel to access said biosensor member.

17. The field effect transistor-based biosensor assembly according to claim 16, wherein said microfluidic channel has an upstream end portion and a downstream end portion, said microfluidic member is formed with an inlet port and an outlet port disposed at said upstream end portion and said downstream end portion of said the microfluidic channel, respectively, to fluidly communicate with said microfluidic channel.

18. The field effect transistor-based biosensor assembly according to claim 11, further comprising an open-well member which defines an open well in the form of an S-shape for accommodating a fluid that contains the bacteria therein, and which is disposed on said biosensors to permit the bacteria in said open well to access said biosensor member.

19. The field effect transistor-based biosensor assembly according to claim 12, further comprising a microfluidic member which defines a microfluidic channel in the form of a circular shape for passage of a fluid containing the bacteria therethrough, and which is disposed on said biosensors to permit the bacteria in said microfluidic channel to access said biosensor member.

20. The field effect transistor-based biosensor assembly according to claim 19, wherein said microfluidic channel has an upstream end portion and a downstream end portion, said microfluidic member is formed with an inlet port and an outlet port disposed at said upstream end portion and said downstream end portion of said microfluidic channel, respectively, to fluidly communicate with said microfluidic channel.

21. The field effect transistor-based biosensor assembly according to claim 12, further comprising an open-well member which defines an open well in the form of a circular shape for accommodating a fluid that contains the bacteria therein, and which is disposed on said biosensors to permit the bacteria in said open well to access said biosensor member.
